(19) United States
(12) Patent Application Publication
(10) Pub. No.: US 2017/0065663 A1
(43) Pub. Date: Mar. 9, 2017

(54) SUBSTANCE AND METHOD FOR TREATING INFLUENZA

(71) Applicants: Michael K. Wilhelm, Scottsdale, AZ (US); Christopher A. Romano, Sandpoint, ID (US)

(72) Inventors: Michael K. Wilhelm, Scottsdale, AZ (US); Christopher A. Romano, Sandpoint, ID (US)

(73) Assignee: New Amsterdam Sciences, Scottsdale, AZ (US)

(21) Appl. No.: 15/308,556

(22) PCT Filed: May 4, 2015
(86) PCT No.: PCT/US15/29043

§ 371 Date: Nov. 2, 2016

Related U.S. Application Data
(60) Provisional application No. 61/988,123, filed on May 2, 2014.

(51) Int. Cl. A61K 38/04; A61K 9/00; A61K 45/06

(52) U.S. Cl. CPC A61K 38/046; A61K 45/06; A61K 9/0053

(57) ABSTRACT

A composition and method for treatment and protecting against influenza that includes bioactive Substance P and/or its analogs. The composition agents can be administered via inhalation therapy, intravenously, intramuscularly, sublingually, or by other methods.

The present application claims priority to U.S. Provisional Application No. 61/988,123 filed May 2, 2014, the specification of which is incorporated in its entirety herein.

### BACKGROUND

The present invention pertains to compounds and methods that have utility for treating and/or preventing and/or mitigating the effects of influenza.

Influenza affects millions of people every year during the annual “flu season”. As influenza viruses infect and transmit between these millions of hosts, the virus undergoes continuous changes and mutations. Currently, the major influenza strains in circulation include the H1N1, H3N2, H5N1 and H7N9 subtypes. These strains differ in transmissibility (the ability of the virus to spread) and pathogenicity (the ability of the virus to cause disease). “Seasonal” influenza strains like common H1N1 or H3N2 are generally highly-transmissible and relatively lowly-pathogenic, whereas “pandemic” or “avian” influenza strains like H5N1 and H7N9 are generally lowly-transmissible and highly-pathogenic. However, the constant mutations of the influenza virus sometimes lead to strains that are both highly transmissible and highly-pathogenic, as was the case with the H1N1 flu of 1918.

While “seasonal” influenza strains often only cause severe illness in at risk populations such as in the elderly, young children and people suffering from other chronic illnesses or conditions, all influenza strains have the capacity to affect people from all demographics in lesser or greater severity. Influenza outbreaks can and do put strains on healthcare delivery systems and have adverse effects on individuals' quality of life and ability to carry on with necessary responsibilities and activities.

Influenza manifests with one or more systemic symptoms from mild to severe. In severe manifestations, the reaction to influenza infection can result in an excessive release of pro-inflammatory cytokines described as a “cytokine storm”. This immunological phenomenon has been implicated as a significant factor in the morbidity and mortality of many pandemic influenza victims. The cytokine storm can cause tissue damage and the accumulation of cell debris that can clog airways leading to decrease in lung function and pneumonia and can contribute to or aggravate various existing cardiopulmonary conditions.

Currently, influenza illness is treated by with antiviral drugs. The Antivirals currently employed function as chemophylactics and/or which work to slow the production of the virus in the body. These treatments are most effective when started within the first 24 hours of illness, a time-point where symptoms of influenza infection may not yet be present. However, recent reports from the Cochrane Collaboration, the benchmark of unbiased high-quality medical treatment reviews, indicate that these antiviral treatments are not broadly-effective and in most cases lead to a reduction in the length of sickness of less than 1 day.

As a result, there is significant demand for new influenza treatments, which are driven by the morbidity and mortality from infections and the identification of new influenza strains. Vaccines and antiviral drugs are both rendered less effective by variability of the influenza virus strain, thus demand is especially great for a treatment that is effective independent of the influenza strain. In contrast to the influenza virus, human physiology does not change quickly. Therefore, drugs that target the immune system to improve its ability to fight influenza infection are particularly appealing

### SUMMARY

According to one embodiment of the invention a material a doseform effective for treating a patient that has been infected or exposed to influenza is disclosed. The dose from includes an effective amount of an agent selected from the group consisting of: substance P, [Met-OH¹¹]-substance P, [Met-OMe¹¹]-substance P, [Nle¹¹]-substance P, [Pro⁹]-substance P, [Sar⁹]-substance P, [Tyr⁸]-substance P, Sar⁹, Met (O₂)¹¹-Substance P, and [p-Cl-Phe^{7,8}]-substance P is administered to the patient. A disease feature is thereby decreased. Also disclosed is a method for treating an individual who has been exposed to influenza which includes administering at least one dose of a material that contains substance P, [Met-OH¹¹]-substance P, [Met-OMe¹¹]-substance P, [Nle¹¹]-substance P, [Pro⁹]-substance P, [Sar⁹]-substance P, [Tyr⁸]-substance P, Sar⁹, Met (O₂)¹¹-Substance P, and [p-Cl-Phe^{7,8}]-substance P

According to another embodiment of the invention as disclosed is directed to a material and doseform that is effective in providing protection which prevents an individual from developing influenza and symptoms related to influenza. The individual has been or is expected to be exposed to a patient with influenza. The doseform includes an effective amount of an agent selected from the group consisting of: substance P, [Met-OH¹¹]-substance P, [Met-OMe¹¹]-substance P, [Nle¹¹]-substance P, [Pro⁹]-substance P, [Sar⁹]-substance P, [Tyr⁸]-substance P, Sar⁹, Met (O₂) 11-Substance P, and [p-Cl-Phe^{7,8}]-substance P. This material can be administered to the individual prior influenza exposure and thereby reduce the risk of developing influenza.

### DESCRIPTION OF THE DRAWINGS

In order to facilitate the present disclosure, reference is made to the following illustrative drawing figures in which like reference numerals are employed where appropriate throughout the various views:

FIG. 1 is a space filling model of Substance P: Arg-Pro-Lys-Pro-Gln-Gln-Phe-Phe-Gly-Leu-Met-NH₂;

FIG. 2 is a structural depiction of an analog of Substance P as disclosed herein;

FIG. 3 is a table depicting the amino acid sequence for Sar⁹, Met (O₂)¹¹-Substance P and endogenous tachykinins

FIG. 4 depicts infected control lungs showing Influenza virions inside infected cells (left picture, arrow) and the lack of airway cilia in infected lung (right picture. single arrow) and stressed airway epithelial cell with numerous mitochondria (right picture, double arrows;

FIG. 5 depicts treated lungs show normal ciliated epithelial cell (single arrow) and normal mitochondrial complement (double arrows);

FIG. 6 depicts viral titers in the nose at both 1 day and 4 days post-infection in animals mock-treated or treated with the compound disclosed herein;

FIG. 7 represent lung viral titers at both 1 day and 4 days post-infection in animals mock-treated or treated with the compound disclosed herein;

FIG. 8 represents. viral titers in the lungs at 1 day post-infection in animals mock-treated, treated with Tamiflu®, treated with Sar⁹, Met (O₂)¹¹-Substance P, or treated with Sar⁹, Met (O₂)¹¹-Substance P plus oseltamivir (Tamiflu®);

FIG. 9 is a graph depicting body weight changes over time in animals mock-treated, treated with Tamiflu®, or treated with Sar⁹, Met (O₂)¹¹-Substance P, or treated with Sar⁹, Met (O₂)¹¹-Substance P plus oseltamivir (Tamiflu®);

FIG. 10 is a graph depicting body weight changes over time in animals mock-treated, treated with Tamiflu®, or treated with Sar⁹, Met (O₂)¹¹-Substance P(2 doses);

FIG. 11 is a graph depicting of data associated with lung inflammation in animals mock-treated, treated with oseltamivir (Tamiflu®), treated with Sar⁹, Met (O₂)¹¹-Substance P, or treated with oseltamivir (Tamiflu®) and Sar⁹, Met (O₂)¹¹-Substance P;

FIG. 12 is a graph depicting animal mortality in animals treated with oseltamivir (Tamiflu®) or treated on Sar⁹, Met (O₂)¹¹-Substance P;

FIG. 13 is a graph depicting clinically-determined sickness scores over time of animals infected with 10̂3 PFU H5N1+/− treated with Sar⁹, Met (O₂)¹¹-Substance P, 10̂5 PFU H5N1+/− treated with Sar⁹, Met (O₂)¹¹-Substance P, and animals not infected but treated with Sar⁹, Met (O₂)¹¹-Substance P;

FIG. 14 is a graph depicting temperature over time of animals infected with 10̂3 PFU H5N1+/− treated with Sar⁹, Met (O₂)¹¹-Substance P, 10̂5 PFU H5N1+/− treated with Sar⁹, Met (O₂)¹¹-Substance P, and animals not infected but treated with Sar⁹, Met (O₂)¹¹-Substance P; and

FIG. 15 is a graph depicting percent survival of animals infected with 10̂3 PFU H5N1+/− treated with Sar⁹, Met (O₂)¹¹-Substance P, 10̂5 PFU H5N1+/− treated with Sar⁹, Met (O₂)¹¹-Substance P, and animals not infected but treated with Sar⁹, Met (O₂)¹¹-Substance P.

### DETAILED DESCRIPTION

It has been discovered that Substance P and/or its bioactive analogs, including but not limited to, Sar⁹, Met (O₂) 11-Substance P, is a beneficial treatment of influenza. Substance P and its analogs also potentiate the lung immune system's response against various major influenza strains including but not limited to H1N1, H3N2, H5N1 and H7N9. Substance P and its analogs can be used to prophylactically treat health care workers and family members who must care for influenza patients and suspected influenza patients.

It has been found, quite unexpectedly that Substance P (RPKPQQFFGLM-NH₂; SEQ ID NO: 1) or a bioactive analog thereof, such as Sar⁹, Met (O₂)¹¹-Substance P, can be administered to treat ARDS, SARS, corona and corona-like respiratory virus infections. As used herein, the term “bioactive analog of Substance P” include various synthetic materials that evidence the ability to interact with and/or agonize the NK-1 receptor. Routine assays for such activities are known in the art and can be used. As disclosed herein, the bioactive analog can be selected from the group that includes [Met-OH¹¹]-substance P, [Met-OMe¹¹]-substance P, [Nle¹¹]-substance P, [Pro⁹]-substance P, [Sar⁹]-substance P, [Tyr⁸]-substance P; Sar⁹, Met (O₂)¹¹-substance P; and [p-Cl-Phe^{7,8}]-substance P.

The dose form containing substance P and/or analogs thereof can be administered to the individual patient to mitigate or prevent influenza infection. The dose form can be configured to administration by various methods known in the art including, but not limited to, aerosol inhalation, intravenous administration, intratracheal administration, intrabronchial administration, intramuscular administration, sublingual administration, and oral administrations. Contemplated intravenous dosages will include 0.05 to 5 nanomolar substance P or substance P analog for suitable intravenous administration. In certain intravenous applications, a dose concentration of 0.1 to 2 nanomolar may be employed; while in other applications 0.5 to 1.5 nanomolar dose concentrations can be used. For aerosol administration contemplated dosages include 0.05 to 5.0 micromolar substance P or analog; with dosages from 0.1 to 2 micromolar employed in certain applications; and dosages from 0.5 to 1.5 micromolar employed in others. Typical concentration ranges of substance P or its bioactive analog in the aerosol administered is between 0.001 and 10 μM. The Substance P or substance P analog can be advantageously administered as a liquid dose form at a concentration between about 0.1 and 10 μM. In certain applications, it is contemplated that the bioactive material disclosed herein will be substance P analogs, while in other applications, it is contemplated that the bioactive composition can be a combination of endogenously derived Substance P and Substance P derivatives.

Thus as broadly disclosed, the composition may include the following: an effective amount of a substance P compound; and a carrier medium, wherein the carrier medium is one of an aqueous solution, a food grade organic liquid, an inert food grade solid and wherein the substance P compound is present in the carrier medium in an amount sufficient to provide a dose strength between 0.05 nanomolar and 10 micromolar. The substance P compound can be at least one of the following: Substance P, Substance P fragments; Sar⁹, Met (O₂)¹¹-Substance P; Sar⁹, Met (O₂)¹¹-Substance P fragments; [Met-OH¹¹]-substance P; [Met-OH¹¹]-substance P fragments [Met-OMe¹¹]-substance P; [Met-OMe¹¹]-substance P fragments; [Nle¹¹]-substance P; [Nle¹¹]-substance P fragments; [Pro⁹]-substance P; [Pro⁹]-substance P fragments; [Sar⁹]-substance P; [Sar⁹]-substance P fragments; [Tyr⁸]-substance P; [Tyr⁸]-substance P fragments; [p-Cl-Phe^{7,8}]-substance P; [p-Cl-Phe^{7,8}]-substance P fragments. In certain applications the substance P compound will be one of the following: Sar⁹, Met (O₂)¹¹-Substance P; [Met-OH¹¹]-substance P; [Met-OMe¹¹]-substance P; [Nle¹¹]-substance P; [Pro⁹]-substance P; [Sar⁹]-substance P; [Tyr⁸]-substance P; [p-Cl-Phe^{7,8}]-substance P.

Bioactive analogs, according to the invention are those which act as competitive inhibitors of Substance P by binding to the Substance P receptor (NK-1 receptor). The analogs may be agonists of the NK-1 receptor. Other derivatives as could be formulated by methods known in the art can be used. Various commercially available materials may also be employed. In addition, substance P fragments and derivatized substance P fragments may also be used. Substitution, deletion, or insertion of one to eight amino acid residues, and preferably from one to three amino acid residues, will lead to analogs which can be routinely tested for biological activity. In addition, functional groups may be modified on Substance P while retaining the same amino acid backbone. Again, routine testing will determine which of such modifications do not adversely affect biological activity. This disclosure contemplates the use of substance P fragments and/or derivitized Substance P fragments having suitable bioactivity in combination with the Substance P materials outlined above. Thus the materials that can be employed include Substance P, Substance P fragments; Sar⁹, Met (O₂)¹¹-Substance P; Sar⁹, Met (O₂)¹¹-Substance P fragments; [Met-OH¹¹]-substance P; [Met-OH¹¹]-substance P fragments [Met-OMe¹¹]-substance P; [Met-OMe¹¹]-substance P fragments; [Nle¹¹]-substance P; [Nle¹¹]-substance P fragments; [Pro⁹]-substance P; [Pro⁹]-substance P fragments; [Sar⁹]-substance P; [Sar⁹]-substance P fragments; [Tyr⁸]-substance P; [Tyr⁸]-substance P fragments; [p-Cl-Phe^{7,8}]-substance P; [p-Cl-Phe^{7,8}]-substance P fragments.

Without being bound to any theory, it is believed that Substance P, the parent compound for the various analogs disclosed herein, is a relatively small (1,348 Daltons), endogenous peptide. The naturally occurring compound was first discovered in 1931 and characterized chemically about 40 years later. Neuropeptides, such as Substance P (SP), when originally discovered were thought to be located and distributed throughout the peripheral and central nervous systems. However, Substance P has since been shown to be produced in non-neuronal cells such as human endothelial cells, Leydig cells, enterochromaffin cells, epithelial cells, fibroblasts, keratinocytes, intestinal and airway smooth muscle cells, inflammatory and immune cells, and in cells of the female reproductive system.

Historically, Substance P has been recognized as a mediator of nonadrenergic, noncholinergic (NANC) excitatory neurotransmission, and as playing a role in the transmission of pain. More recent studies have called into question Substance P's role in pain and pain perception, however, and Substance P antagonists are believed to not affect pain or pain perception.

Modern research has brought forth additional functions for Substance P including the following: vasodilation, smooth muscle contraction, submucosal gland secretion; increased vascular permeability; stimulation of mast cells, B- and T-lymphocytes and macrophages; modulation of chemo-attraction of eosinophils and neutrophils, and modulation of vascular adhesion neutrophils. The ability of Substance P to impact immune cells is of special interest given the immune-modulating properties for one or more of the various analogs.

It is believed that Substance P and the analogs outlined herein binds to one or more of the three neurokinin receptors (NK-1, NK-2 and NK-3), though Substance P preferentially interacts at the NK-1R to mediate its biological effects. The neurokinin receptors belong to “family 1” (rhodopsin-like) of the G protein-coupled receptors. Like many G protein-coupled receptors, the NK-1R consists of seven putative a-helical transmembrane segments, an intracellular carboxyl tail, and an extracellular amino-terminus. At the extracellular amino terminus there is an N-glycosylation site, while many serine and threonine residues at the intracellular carboxyl terminus are potential phosphorylation sites.

The Neurokinin-1 receptor has been identified in stem cell lines as well as cells derived from human placental cord blood, rich in hematopoietic stem and progenitor cells. The NK-1R has been identified in a various tissues and cell type and is expressed in immune cells such as T and B lymphocytes, monocytes/macrophages, neutrophils, and mast cells. Non-immune cells like vascular endothelial cells, bone marrow stromal cells, muscle cells, astrocytes, adipocytes, keratinocytes, and fibroblasts also express the NK-1R. The NK-1R receptor is appears to be involved in a number of physiological systems that may be of significance to the immune system, and the cells that support the immune system.

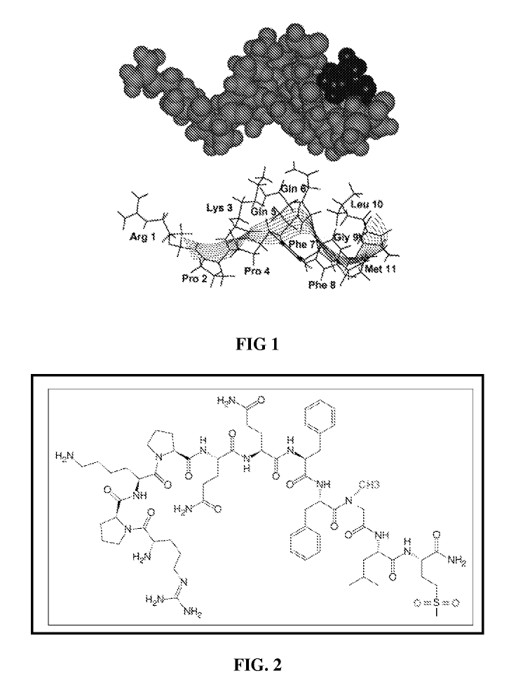

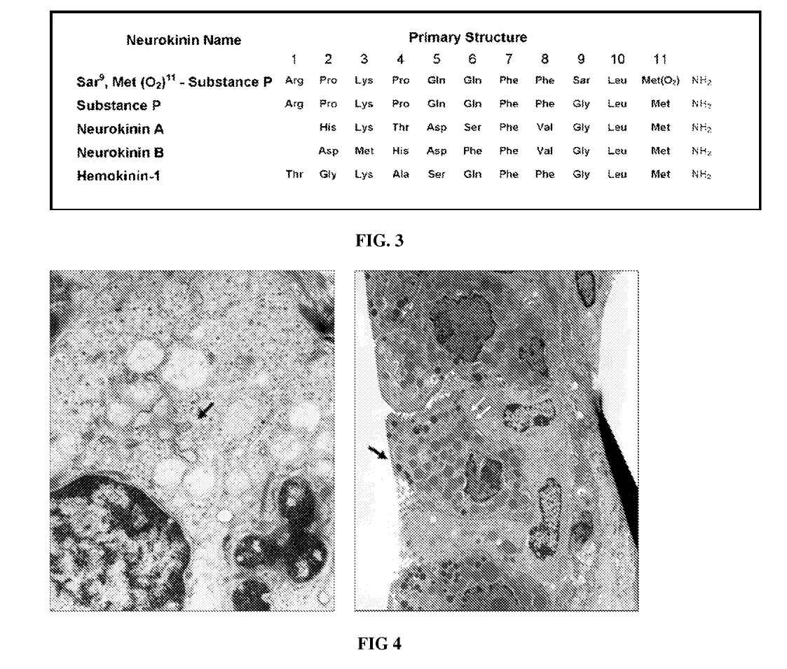

The primary amino acid sequences of Substance P and Sar⁹, Met (O₂)¹¹-Substance P are distinct from the tachykinins Neurokinin A (NKA) and Neurokinin B (NKB) at the N-terminus (referenced by the start of the amino acid sequence as seen in FIG. 3. These differences result in NKA and NKB binding with significantly less affinity to the Neurokinin-1 receptor (NK-1R) than do SP and Sar⁹, Met (O₂)¹¹-Substance P, which share an identical N-terminal sequence. The C-terminal penta-peptide, Phe-Phe/Val-Gly-Leu-Met-NH₂, is conserved between all natural tachykinins and is required for receptor activation. Amidation of the C-terminal methionine is vital for peptide function as without this modification tachykinins are unable to activate their corresponding mammalian receptors. The specific C-terminal modifications that separate Sar⁹, Met (O₂)¹¹-SP from endogenous Substance P are believed to contribute to differences in bioactivity and confer NK-1 receptor specificity (via the modification of the Gly⁹), as the ligand-receptor interactions are changed as depicted in FIG. 2.

By way of non-limiting example, one particular analog that has been found to be particularly efficacious in treatment of influenza is created by modifying two of the eleven amino acids in the Substance P sequence. These modifications included replacing glycine (Gly) with sarcosine (Sar or N-methyl glycine) at the ninth position and introducing an oxidized form of methionine [Met(O₂)] at the eleventh position (as seen in FIG. 2 and resulting in the peptide Sar⁹, Met (O₂)¹¹-SP (1,393.6 Daltons)

Without being bound to any theory, it is believed that materials such as Sar⁹, Met (O₂)¹¹-Substance P and the analogues disclosed herein mediate predominately through interactions with the Neurokinin-1 receptor located on the plasma membrane of many cell types. Substance P binds to all three neurokinin receptors (NK-1, NK-2 and NK-3), though Substance P preferentially interacts at the NK-1R to mediate its biological effects. Compounds such as Sar⁹, Met (O₂)¹¹-Substance P, [Met-OH¹¹]-Substance P, [Met-OMe¹¹]-Substance P, [Nle¹¹]-Substance P, [Pro⁹]-Substance P, [Sar⁹]-Substance P, [Tyr⁸]-Substance P, [p-Cl-Phe^{7,8}]-Substance P and the like are believed to be highly selective for NK-1R while demonstrating little or no activity in NK-2 and NK-3 receptor biological activity assays while eliciting greater than 3-times the biological activity as naturally occurring Substance P in NK-1R-specific tissues. The two-fold greater binding affinity for analogs such as Sar⁹, Met (O₂)¹¹-SP shows for the NK-1R compared to naturally occurring Substance P (0.8±0.3 nM vs. 1.6±0.4 nM, respectively) is evidence of increases in biological activity

The interaction of both Sar⁹, Met (O₂)¹¹-Substance P and naturally occurring Substance P with the NK-1R induces secondary messenger signaling events, which originate from the activated receptor and then rapidly cascade throughout the cell. Water-soluble messengers, like Ca²⁺ and cyclic AMP (cAMP), diffuse throughout the cytosol, while the hydrophobic lipid-soluble messengers like diacylglycerol (DAG) diffuse into the plasma membrane. In some tissues, activation of water-soluble messengers can lead to signaling events in adjacent cells via gap junctions, thus leading to a broad multi-cellular response from the stimulation of a single cell

Substance P and analogues such as Sar⁹, Met (O₂)¹¹-SP are capable of utilizing both phosphatidylinositol (PI) hydrolysis and cAMP as second messenger signaling systems (mediating changes in intracellular Ca²⁺ mobilization), and do so with high potency. Using an in vitro model (Chinese Hamster Ovary cells transfected with human NK-1R) Sagan et al. have reported an EC₅₀ of 1.0±0.6 nM for Substance P in stimulating phosphatidylinositol (PI) hydrolysis whereas Sar⁹, Met (O₂)¹¹-Substance P was reported to have an EC₅₀ of 0.40±0.01 nM in stimulating PI hydrolysis, Sagan, S. et al., J. Pharmacology and Experimental Therapeutics 276 (1996) 1039-1048. The same group reported an EC₅₀ of 8±4 nM for Substance P in stimulating cAMP formation whereas Sar⁹, Met (O₂)¹¹-Substance P was reported to have an EC₅₀ of 16±7 nM.

The Substance P analog, Sar⁹, Met (O₂)¹¹-Substance P, is a 1393 Da, 11-amino acid, synthetically-manufactured analog of Substance P (SP). Sar⁹, Met (O₂)¹¹-Substance P is modified at the 9th [N-Methyl glycine (or Sarcosine) instead of glycine] and 11th (addition of a Sulphone) positions. These modifications render Sar⁹, Met (O₂)¹¹-Substance P receptor-specific for the Neurokinin-1 receptor, and also make Sar⁹, Met (O₂)¹¹-Substance P more resistant to proteolytic degradation relative to the endogenous peptide, Substance P.

The differences in the degradation characteristics between the endogenous peptide Substance P and Sar⁹, Met (O₂)¹¹-Substance P have recently been found to result in different biological effects. Ligands that signal via G protein-coupled receptors elicit downstream cellular signals (e.g. cAMP NF-κB, etc.), which consequently produce a physiological effect. Once the ligand binds the receptor (the NK1 receptor in the case of Sar⁹, Met (O₂)¹¹-Substance P and Substance P), downstream cellular signals are elicited and the ligand-receptor complex is internalized from the plasma membrane into an early endosome, where the ligand is degraded. Recently Cattaruzza et al. reported that the endopeptidase called endothelin-converting enzyme-1 (ECE-1) is responsible for the degradation of internalized neuropeptides within early endosomes, Cattaruzza, F. et al., Br J Pharmacol. 2009 March; 156(5):730-9. ECE-1 shares considerable sequence homology with Neprilysin (a.k.a. Neutral Endopeptidase or CD10), which is recognized for degrading neuropeptides extracellularly. Indeed, the CD10/CALLA (common acute lymphoblastic leukemia antigen) nomenclature reflects the historical localization of the enzyme's activity on the surface of intact cells. ECE-1 hydrolyzes SP at Gln⁶-Phe⁷ and Gly⁹-Leu¹⁰ linkages and degradation of endocytosed neuropeptides regulates trafficking and signaling of internalized receptors. This enzyme facilitates the release of the receptor from the ligand-receptor complex and allows for the receptor to be recycled back to the cell surface, which mediates resensitization.

It is believed that the ability of the ligand to be degraded by ECE-1 is critical to the resensitization of the cell following activation. In the case of Substance P, this occurs rapidly and efficiently. However in the case of Sar⁹, Met (O₂)¹¹-Substance P, this process is considerably less efficient. It is believed that Sar⁹, Met (O₂)¹¹-Substance P is degraded significantly slower and less efficiently than the endogenous tachykinin Substance P: within 100 minutes of incubation in a solution containing 195 nM ECE-1, nearly 100% of the Substance P present is degraded. However, at this same concentration of ECE-1 only about 20% of the Sar⁹, Met (O₂)¹¹-Substance P present is degraded. In fact, it takes nearly 300 minutes for 50% of the Sar⁹, Met (O₂)¹¹-Substance P present to become degraded and no additional degradation occurs even at 1200 minutes post-incubation. It is believed that within NK1-R expressing cells ECE-1 degradation of Substance P disrupted the Substance P-NK1R association with β-arrest in-Src complexes and the resultant ERK1/2 activation. Thus the effect of a slower degradation of Sar⁹, Met (O₂)¹¹-Substance P and its complex with internalized NK1R might result in prolonged activation of intracellular ERKs (extracellular signal-regulated kinases) and a different (prolonged ERK activation but delayed receptor resensitization), perhaps enhanced cellular response compared to that elicited by endogenous Substance P

Without being bound to any theory, it is believed that Sar⁹, Met (O₂)¹¹-Substance P may induce cellular responses that differ from those elicited by Substance P and reduce the rate at which NK1 receptor recycling and resensitization occurs. The physiological implications of this recent finding are many, and could include a greater initial stimulatory response to NK1R stimulation due to the prolonged intracellular kinase activation followed by a prolonged resistance to endogenous Substance P due to the inhibition of NK1R recycling.

As disclosed herein, Substance P analogues have particular applicability as a primary or adjunct therapeutic material for treatment of influenza. Suitable analogues include but are not limited to [Met-OH¹¹]-substance P, [Met-OMe¹¹]-substance P, [Nle¹¹]-substance P, [Pro⁹]-substance P, [Sar⁹]-substance P, [Tyr⁸]-substance P, Sar⁹, Met (O₂)¹¹-substance P, and [p-Cl-Phe^{7,8}]-substance P. It is believed that the various analogs as outlined will exhibit similar enhanced characteristics and performance as compared to endogenous Substance P as discussed with Sar⁹, Met (O₂)¹¹-Substance P. The Substance P analogs disclosed herein can be formulated into a suitable dose form that can be administered to prevent or ameliorate one or more symptoms associated with influenza infection.

The Substance P analog material can be administered in a concentration sufficient to active bioactive effect in the patient to whom the material is administered. Bioactive effect, as the term is employed herein, is taken to mean a dose response exhibited in an average of 50% of the symptomatic or pre-symptomatic individuals to be treated. Dose response as defined herein can include but are not limited to one the following: reduction in fever; improved lung histopathology expressed as interstitial pathology and/or alveolitis; reduced lung viral titers, and the like.

In various embodiments, the material disclosed herein can be formulated as an ingestible liquid composed of a carrier material with active component present in a concentration amount between 0.1 and 10 μM. The carrier liquid can be an aqueous solution that may include various short chain food grade alcohol materials, flavoring agents, and the like. Where desired or required, the liquid dose form can also include suitable active compounds including but not limited to analgesic or anti-inflamatory compounds.

It is also contemplated that the bioactive analog Substance P component can be formulated as a solid dose form as a pill or tablet or as a powder or granular material. In certain applications in which the dose form employed is a pill or powder, it is contemplated that the bioactive analog of Substance P disclosed herein can be present in the dose form at a dose concentrations between 0.05 to 7.0 nanomolar. In certain applications, it is contemplated that the active compound disclosed herein can be present at suitable therapeutic concentrations, for example 0.05 to 5.0 nanomolar. The pill or tablet can be formulated with suitable inert carrier materials as desired or required. The pill or table can also include complementary active agents such as analgesics anti-inflammatory materials and the like

The aerosolizable material can be present as a soluble aerosolizable particulate or as an aerosolizable liquid present in the carrier material in a concentration amount between 0.1 and 10 μM. Suitable devices for administering the aerosol of the present invention include nebulizers as well as hand-held aerosol “puffer” devices.

Suitable treatment regimens for treatment and administration of the dose form according to the present disclosure include daily or multiple daily treatment by aerosol. Other modes of treatment include continual transdermal infusion, intravenous injection, intramuscular, sublingual, subcutaneous injection, and oral administration. Suitable formulations of the substance P analogs disclosed herein for administration are any which are pharmaceutically acceptable and in which substance P analog retains its biological activity. Generally, such formulations include substance P analogs dissolved in normal sterile saline solution. Other formulations for changing absorption and half-life characteristics can be used, including liposomal formulations and slow-release formulations.

### Example I

The degradation characteristics of Sar⁹, Met (O₂)¹¹-Substance P relative to unmodified endogenously derived Substance P were studied. Samples Substance P and Sar⁹, Met (O₂)¹¹-Substance P were incubated at equivalent concentrations in IMDM media with 2.5% fetal bovine serum for 72 hours. Only 24% of the initial concentration of endogenously derived Substance P was recovered at this time whereas 59% of the initial concentration of Sar⁹, Met (O₂)¹¹-Substance P was recovered. This recovery was 146% greater than the amount of endogenously derived Substance P recovered. This demonstrates that the Sar⁹, Met (O₂)¹¹-Substance P is significantly less than prone to degradation than endogenously derived Substance P.

### Example II

The degradation characteristics of the following Substance P analogs relative to unmodified endogenously derived Substance P are studied: [Met-OH¹¹]-substance P; [Met-OMe¹¹]-substance P; [Nle¹¹]-substance P; [Pro⁹]-substance P; [Sar⁹]-substance P; [Tyr⁸]-substance P; [p-Cl-Phe^{7,8}]-substance P. Unmodified Substance P and the various enumerated Substance P analogs are incubated at equivalent concentrations in IMDM media with 2.5% fetal bovine serum for 72 hours. Only 24% of the initial concentration of endogenously derived Substance P is recovered at this time whereas greater than 50% of the initial concentration of the various enumerated substance P analogs is recovered. This recovery is at least 100% greater than the amount of endogenously derived Substance P recovered. This demonstrates that the enumerated Substance P analogs are significantly less than prone to degradation than endogenously derived Substance P.

### Example III

The reactions effected by Sar⁹, Met (O₂)¹¹-Substance P in animals infected with influenza were studied with one aspect of the pathology. One aspect of the pathology of influenza is a localized inflammatory response in the lungs, which under certain conditions (such as infection by highly pathogenic H5N1 influenza) can lead to large-scale release of numerous inflammatory cytokines. This is a condition termed ‘cytokine storm,’ and results in significant morbidity and mortality. Sar⁹, Met (O₂)¹¹-Substance P has been observed to produce a decrease in the inflammatory state of the lungs in animals infected with influenza following treatment. Specifically demonstrated is a reduction in interstitial inflammation and alveolitis. Thus it is possible that Sar⁹, Met (O₂)¹¹-Substance P can behave in a manner more characteristic of Substance P antagonists. It is believed that this occurs via reducing the NK1 receptor recycling rate and the resensitization of the receptor following receptor activation.

### Example IV

The reactions effected by the following Substance P analogs in animals infected with influenza are studied with one aspect of the pathology: [Met-OH¹¹]-substance P; [Met-OMe¹¹]-substance P; [Nle¹¹]-substance P; [Pro⁹]-substance P; [Sar⁹]-substance P; [Tyr⁸]-substance P; [p-Cl-Phe^{7,8}]-substance P. Sar⁹, Met (O₂)¹¹-Substance P. One aspect of the pathology of influenza is a localized inflammatory response in the lungs, which under certain conditions (such as infection by highly pathogenic H5N1 influenza) can lead to large-scale release of numerous inflammatory cytokines. This is a condition termed ‘cytokine storm,’ and results in significant morbidity and mortality. The enumerated Substance P analogs are observed to produce a decrease in the inflammatory state of the lungs in animals infected with influenza following treatment. Specifically demonstrated is a reduction in interstitial inflammation and alveolitis. Thus it is possible that the enumerated Substance P analogs can behave in a manner more characteristic of Substance P antagonists. It is believed that this occurs via reducing the NK1 receptor recycling rate and the resensitization of the receptor following receptor activation.

### Example V

Cohorts of mice, cotton rats and ferrets were infected with influenza virus infection of one of the following subtypes: H1N1, H3N2, H5N1 and H7N9. The various infected cohorts were divided into a placebo group, a group treated with intranasally administered Sar⁹, Met (O₂)¹¹-Substance P, a group treated with oseltamivir and a group treated with oseltamivir and intranasally administered Sar⁹, Met (O₂)¹¹-Substance P. Subjects treated with intranasally administered Sar⁹, Met (O₂)¹¹-Substance P alone have been observed to exhibit fewer flu-related symptoms than influenza-infected animals treated with a placebo in that clinical symptoms (temperatures and body weights), virus titers, and pulmonary inflammation were reduced. The cohorts treated with oseltamivir exhibited fewer flu-related symptoms than the influenza-infected animals treated with a placebo but did not exhibit the symptom reduction produced in the subjects treated with Sar⁹, Met (O₂)¹¹-Substance P.

### Example VI

Cohorts of mice, cotton rats and ferrets are each infected with an influenza virus infection of one of the following subtypes: H1N1, H3N2, H5N1 and H7N9. The various infected cohorts are divided into a placebo group, a group treated with intranasally one of the following Substance P analogs: [Met-OH¹¹]-substance P; [Met-OMe¹¹]-substance P; [Nle¹¹]-substance P; [Pro⁹]-substance P; [Sar⁹]-substance P; [Tyr⁸]-substance P; [p-Cl-Phe^{7,8}]-substance P. Sar⁹, Met (O₂)¹¹-Substance P. Other groups include a group treated with oseltamivir and groups treated with oseltamivir and one of the enumerated Substance P analogs intra nasally administered. Subjects treated with intranasally administered one of the enumerated Substance P analogs alone are observed to exhibit fewer flu-related symptoms than influenza-infected animals treated with a placebo in that at least one of the following: clinical symptoms (temperatures and body weights), virus titers, and pulmonary inflammation are reduced. The cohorts treated with oseltamivir exhibited fewer flu-related symptoms than the influenza-infected animals treated with a placebo but do not exhibit the symptom reduction produced in the subjects treated with one of the enumerated Substance P analogs.

### Example VII

Mice, cotton rats and ferrets were treated with intranasally administered Sar⁹, Met (O₂)¹¹-Substance P after influenza virus infection with one of the following subtypes: H1N1, H3N2, H5N1 and H7N9 together with treatment with oseltamivir. The test subjects have repeatedly been observed to exhibit fewer flu-related symptoms than influenza-infected animals treated with placebo. Clinical symptoms (temperatures and body weights), virus titers, and pulmonary inflammation are all reduced by Sar⁹, Met (O₂)¹¹-Substance P.

### Example VIII

### Sar⁹, Met (O₂)¹¹-Substance P Treatment in JP-8-Exposed and H3N2-Infected Mice

4-week old female C57BL/6 mice in a 7-day exposure were exposed to a 1000 mg/m³ dose of JP-8 jet fuel, The JP-8 jet fuel aerosol/vapor mixture was drawn through a nose-only mouse exposure chamber using a constant vacuum flow rate of 2.5 liters/minute. After 7 days of a 1 hr/day exposure to JP-8 jet fuel, the mice were infected with a 10 μL aliquot of 2.0×10³ viral titer level of (H3N2) A/Hong Kong/8/68 mouse-adapted influenza virus while under light anesthesia. Animals to be treated with Sar⁹, Met (O₂)¹¹-Substance P were given a 15-minute aerosol treatment of 1 μM Sar⁹, Met (O₂)¹¹-Substance P in sterile, normal saline using an ultrasonic nebulizer (DeVilbiss Model 99) each day following the A/Hong Kong/8/68 inoculation. The A/Hong Kong/8/68-infected control mice were handled in the same manner as the Sar⁹, Met (O₂)¹¹-Substance P-treated mice except for the daily Sar⁹, Met (O₂)¹¹-Substance P treatment.

On Day 5 following viral infection, the infected mice exhibited obvious signs of respiratory illness including wheezing, labored breathing, and mucus accumulation in their nasal passages. On Day 7 after viral infection, the infected mice began to die of Acute Respiratory Distress Syndrome (ARDS) and surviving Sar⁹, Met (O₂)¹¹-Substance P-treated and control animals entered the studies described below. Note that at Day 7 the Sar⁹, Met (O₂)¹¹-Substance P-treated mice infected with influenza all appeared healthy and did not exhibit any signs of respiratory illness or the development of ARDS

Broncho-alveolar lavage revealed that the Sar⁹, Met (O₂)¹¹-Substance P-treated mice had normal lung cell counts [10×10⁴ cells/ml±0.18×10⁴ (mean±SEM)] while the A/Hong Kong8/68-infected mice demonstrated a very high lung cell count [2.37×10⁶ cells/ml±0.65×10⁶]. It is hypothesized that this large increase in BAL-derived cells could be due to neutrophil chemotaxis driven by macrophage-released chemotactic factors in the infected animal lungs, although differential cell counts were not performed.

In support of this hypothesis, Sar⁹, Met (O₂)¹¹-Substance P-treated mice had a 67% reduction in the concentration of leukotriene B4 in their broncho-alveolar lavage fluid compared to the control influenza-infected mice. Leukotriene B4 is a well-known chemoattractant for inflammatory cell influx into the lungs, and the reduction suggests Sar⁹, Met (O₂)¹¹-Substance P dampened the inflammatory response in treated lungs, appropriately so if the cell counts were normal and there were no clinical infection, as suggested by the lack of respiratory illness or ARDS

Pathological analysis of electron micrographs demonstrated an absence of normal airway cilia, swollen airway epithelial cells with large numbers of mitochondria, and colonies of A/Hong Kong/8/68 virions throughout the lungs in the influenza-infected mice (see electron micrographs). In contrast, no pathological evidence of lung injury in the Sar⁹, Met (O₂)¹¹-Substance P-treated mice was observed. This finding is consistent with prevention of infection by treatment with Sar⁹, Met (O₂)¹¹-Substance P.

### Example IX

### Pre- and Post-Infection Sar⁹, Met (O₂)¹¹-SP Treatment in H3N2-Infected Cotton Rats

Young adult (6-8 weeks old) cotton rats (Sigmodon Hispidus) of both genders were obtained from an inbred colony maintained at Virion Systems, Inc. (Rockville, Md.). A stock of Influenza/A/Wuhan/359/95 was prepared by Novavax, Inc. (Rockville, Md.) from supernatants of MDCK cells that had been inoculated 3 days previously at a low multiplicity of infection (m.o.i). Animals were grouped into untreated/uninfected controls, untreated/infected, Sar⁹, Met (O₂)¹¹-Substance P (0.023 mg/kg-10 uM×0.1 mL)/infected, Sar⁹, Met (O₂)¹¹-Substance P (0.23 mg/kg-100 uM×0.1 mL)/infected, Sar⁹, Met (O₂)¹¹-Substance P (1.16 mg/kg-500 uM×0.1 mL)/infected. Animals were either treated intranasally with Sar⁹, Met (O₂)¹¹-Substance P or vehicle 1 day before influenza infection OR given Sar⁹, Met (O₂)¹¹-Substance P (0.23 mg/kg-100 uM×0.1 mL) beginning 1 hour following influenza infection. Sar⁹, Met (O₂)¹¹-Substance P treatment was continued daily for 10 days following infection

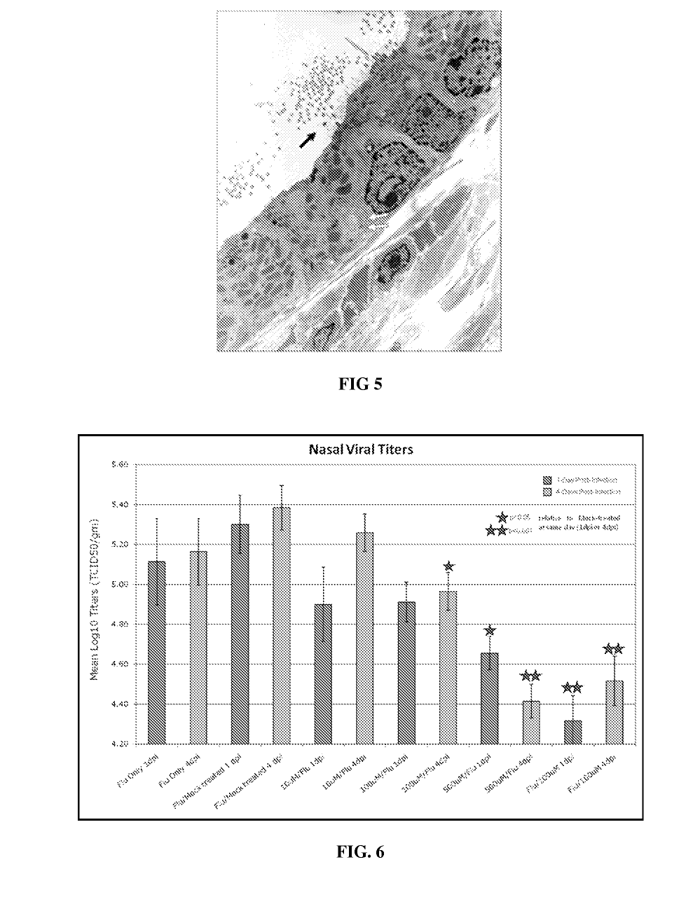

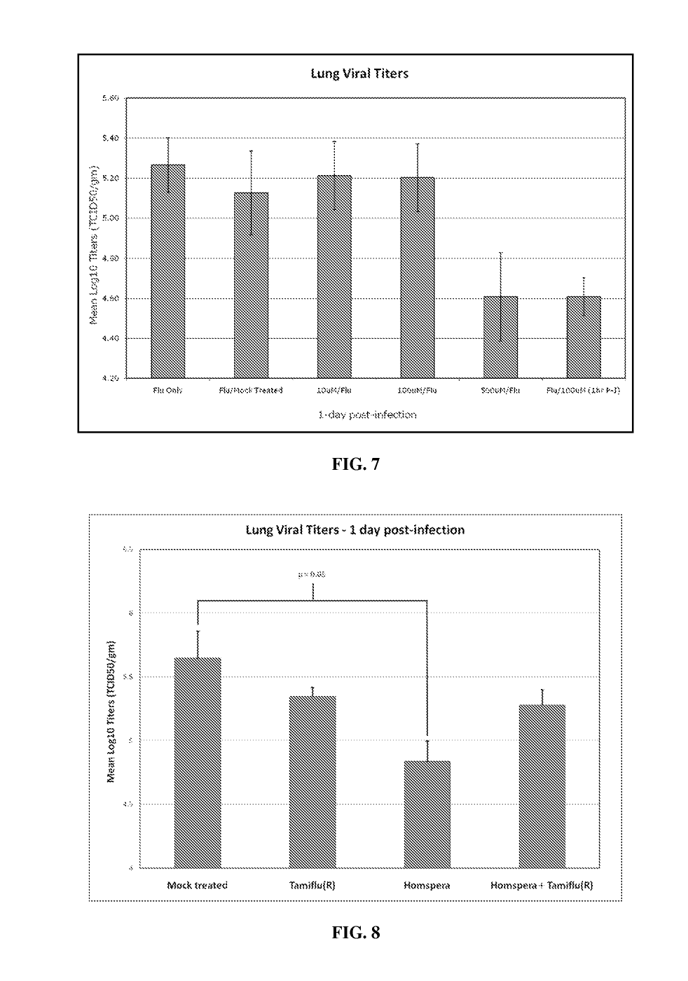

As presented in the FIGS. 6 and 7, statistically-significant (p<0.05) reductions in nasal viral titers were observed in animals treated with 0.23 mg/kg Sar⁹, Met (O₂)¹¹-Substance P prior to influenza infection at 4 days post-infection. Animals treated with 5-fold more Sar⁹, Met (O₂)¹¹-Substance P (1.16 mg/kg) were found to have statistically-significantly reduced nasal viral titers at both 1- and 4-days post-infection. These animals were also observed to have reduced lung viral titers; however the findings did not achieve statistical significance. Animals treated with Sar⁹, Met (O₂)¹¹-Substance P (0.23 mg/kg) following influenza infection were observed to have the greatest reduction in nasal viral titers 1-day following infection and a similar reduction was observed at 4-days post-infection as well—nearly a 90% decrease relative to infected and non-Sar⁹, Met (O₂)¹¹-Substance P-treated controls. Interestingly, the animals treated with Sar⁹, Met (O₂)¹¹-Substance P (0.23 mg/kg) following infection were also observed to have minimal (less than 3%) body weight loss during the infection time course, whereas infected controls were observed to lose approximately 8% of their body weight during the same time period.

### Example X

### Sar⁹, Met (O₂)¹¹-SP+/−Tamiflu in H3N2-Infected Cotton Rats

Young adult (6-8 weeks old) cotton rats (Sigmodon Hispidus) of both genders were obtained from an inbred colony maintained at Virion Systems, Inc. A stock of Influenza/A/Wuhan/359/95 was prepared by Novavax, Inc. from supernatants of MDCK cells that had been inoculated 3 days previously at a low multiplicity of infection (m.o.i). Tamiflu® (oseltamivir, Roche Inc.) was obtained commercially. Animals were grouped into mock-treated/infected, oseltamivir (Tamiflu®) 2 mg/kg/infected, Sar⁹, Met (O₂)¹¹-Substance P 0.23 mg/kg/infected, oseltamivir (Tamiflu®) 2 mg/kg+Sar⁹, Met (O₂)¹¹-Substance P 0.23 mg/kg/infected. Sar⁹, Met (O₂)¹¹-Substance P was administered intranasally and oseltamivir solution was administered orally. All treatments began approximately 1-hour following infection with Influenza/A/Wuhan/359/95. Sar⁹, Met (O₂)¹¹-Substance P was administered once daily, whereas oseltamivir was administered twice daily, once in the morning and once in the evening. Treatment was continued daily for three days.

As presented in the figures below, nearly statistically-significant (p=0.05) reductions in nasal viral titers at 1-day post-infection were observed in animals treated with 0.23 mg/kg. Sar⁹, Met (O₂)¹¹-Substance P. Tamiflu® administration resulted in reductions in nasal viral titers as well; however these reductions were not as large as the reductions observed in the Sar⁹, Met (O₂)¹¹-Substance P-treated animals. Sar⁹, Met (O₂)¹¹-Substance P plus Tamiflu® treatment resulted in slightly lower nasal viral titers than Tamiflu® treatment alone. Coinciding with reductions in nasal viral titers, animals treated with Sar⁹, Met (O₂)¹¹-Substance P were observed to have the least decrease in body weight following infection. Surprisingly, animals-treated with Tamiflu® were observed to have a greater loss in body weight than mock-treated/infected animals. Sar⁹, Met (O₂)¹¹-Substance P treatment did not positively impact body weight losses seen in Tamiflu®-treated animals, as animals receiving. Sar⁹, Met (O₂)¹¹-Substance P and Tamiflu® were observed to have similar, if not greater, losses in body weight relative to Tamiflu®-treated animals.

### Example XI

### Sar⁹, Met (O₂)¹¹-Substance P with or without Tamiflu in H3N2-Infected Cotton Rats

### Expanded Study

Young adult (6-8 weeks old) cotton rats (Sigmodon Hispidus) of both genders were obtained from an inbred colony maintained at Virion Systems, Inc. A stock of Influenza/A/Wuhan/359/95 was prepared by Novavax, Inc. from supernatants of MDCK cells that had been inoculated 3 days previously at a low multiplicity of infection (m.o.i). Animals were grouped into uninfected, mock-treated/infected, oseltamivir (Tamiflu®) 10 mg/kg/infected, infected/oseltamivir (Tamiflu®) 10 mg/kg, Sar⁹, Met (O₂)¹¹-Substance P 0.23 mg/kg/infected, Sar⁹, Met (O₂)¹¹-Substance P 0.70 mg/kg, oseltamivir (Tamiflu®) 10 mg/kg+Sar⁹, Met (O₂)¹¹-Substance P 0.23 mg/kg/infected, oseltamivir (Tamiflu®) 10 mg/kg+Sar⁹, Met (O₂)¹¹-Substance P 0.70 mg/kg/infected, oseltamivir (Tamiflu®) 10 mg/kg+Sar⁹, Met (O₂)¹¹-Substance P 0.23 mg/kg/infected with Sar⁹, Met (O₂)¹¹-Substance P pre-treatment, and oseltamivir (Tamiflu®) 10 mg/kg+Sar⁹, Met (O₂)¹¹-Substance P 0.70 mg/kg/infected with Sar⁹, Met (O₂)¹¹-Substance P pre-treatment was administered intranasally and oseltamivir solution was administered orally. All treatments began approximately 4-hour following infection with Influenza/A/Wuhan/359/95, unless Sar⁹, Met (O₂)¹¹-Substance P pre-treatment was indicated—which occurred 1-hour prior to infection. Sar⁹, Met (O₂)¹¹-SP was administered once daily, whereas oseltamivir was administered twice daily, once in the morning and once in the evening. Treatment was continued daily for five days

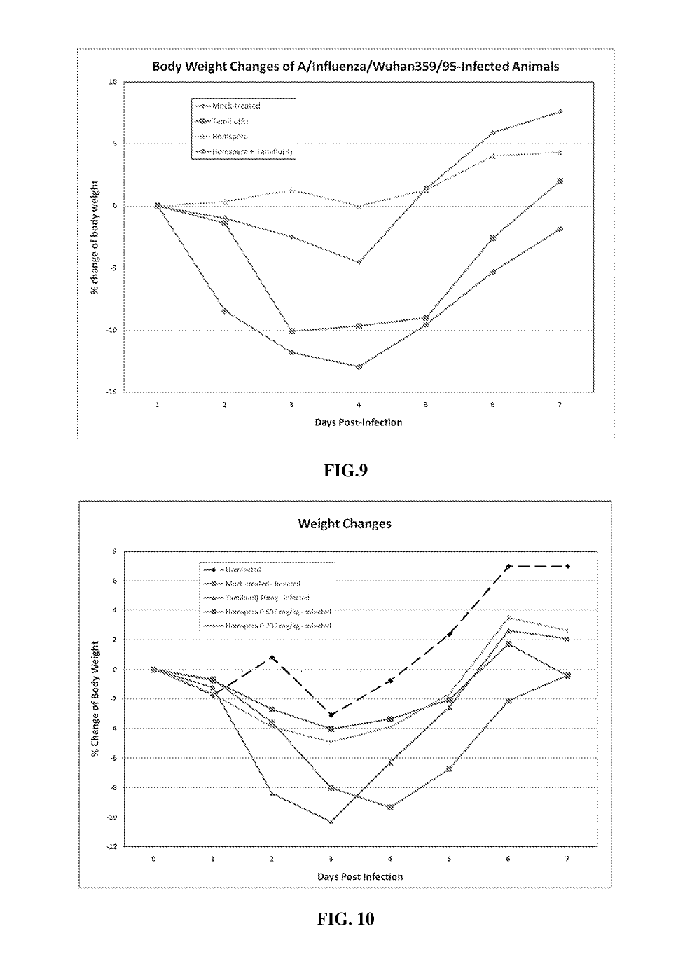

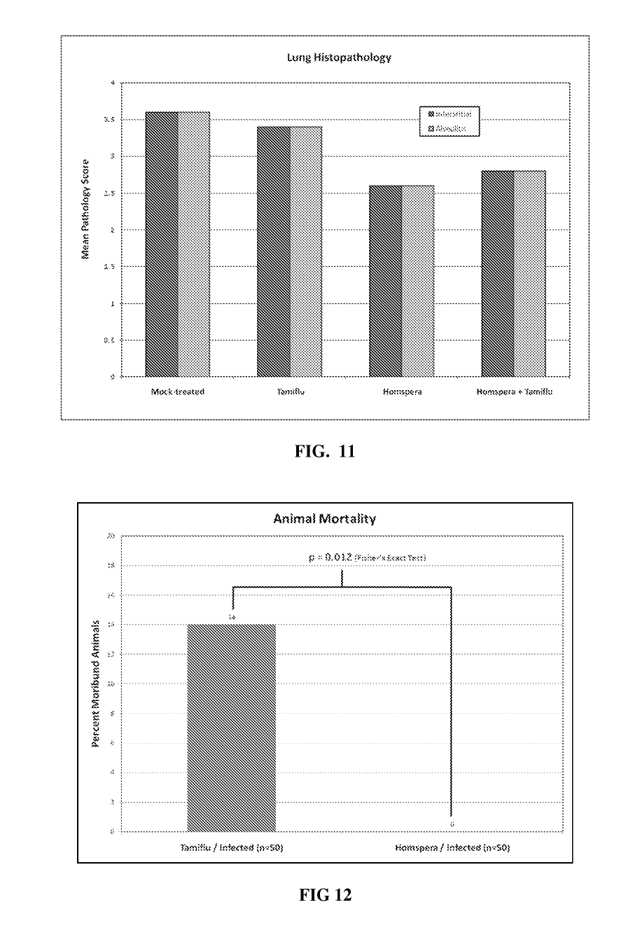

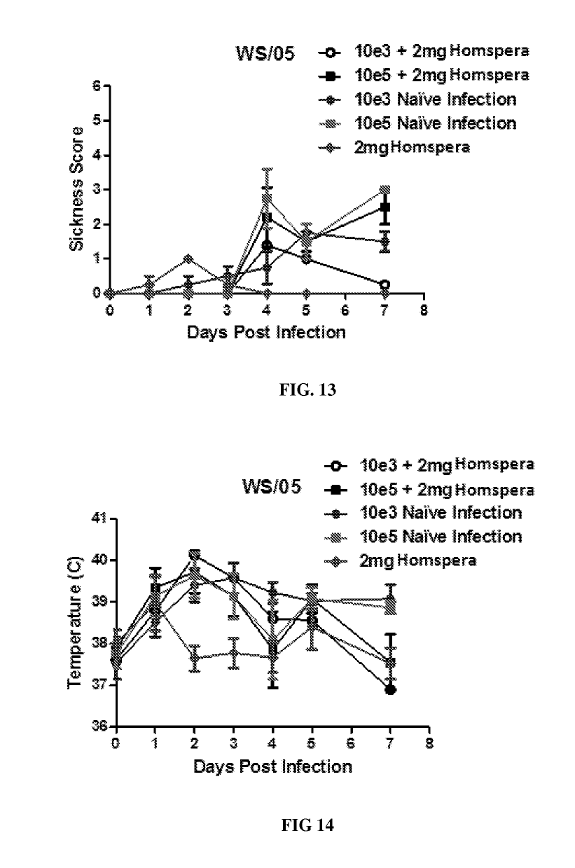

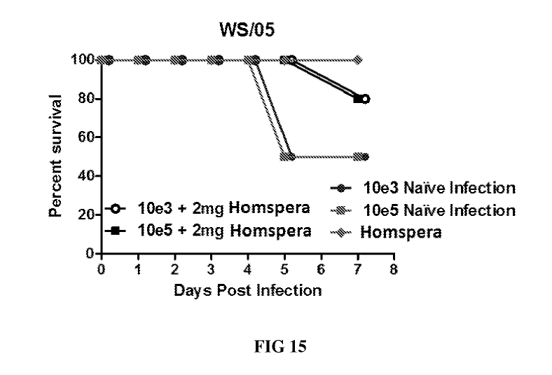

As presented in the FIGS. 10 and 11, animals infected with Influenza/A/Wuhan/359/95 and treated with Sar⁹, Met (O₂)¹¹-Substance P Sar⁹, Met (O₂)¹¹-SP were found to have less body weight loss relative to infected animals treated with Tamiflu® or a control vehicle. The dose of Sar⁹, Met (O₂)¹¹-Substance P treatment (0.23 or 0.70 mg/kg) did not significantly affect animal weight loss. Animals treated with Tamiflu® appeared to recover from weight-loss quicker than vehicle-treated controls, typical of published reports of Tamiflu®

Animals treated with Sar⁹, Met (O₂)¹¹-Substance P were also found to have lower levels of pulmonary inflammation (alveolitis and interstitial inflammation) at 4 days post-infection. Interestingly, Tamiflu®-treatment did not appear to affect pulmonary inflammation in these animals. Sar⁹, Met (O₂)¹¹-Substance P-treatment in addition to Tamiflu® was not as effective as Sar⁹, Met (O₂)¹¹-Substance P treatment alone, suggesting Tamiflu reduces the anti-inflammatory impact of Sar⁹, Met (O₂)¹¹-Substance P, however this effect was slight.

### Example XII

### Sar⁹, Met (O₂)¹¹-Substance P-Treatment in H5N1-Infected Ferrets

A study was performed using a small number of animals to evaluate the potential for Sar⁹, Met (O₂)¹¹-Substance P in mitigating the deleterious effects of highly-pathogenic H5N1 influenza infection.

20 young adult (20 weeks old) Fitch ferrets of both genders were inoculated with Influenza A/Whooperswan/Mongolia/244/2005 (H5N1) with 0, 10³ or 10⁵ plaque-forming units (PFU) on Day 0. Uninfected controls were treated with 2 mg/kg Sar⁹, Met (O₂)¹¹-Substance P via intranasal administration beginning on Day 1 and continued daily thru Day 5. Animals infected with 10³ PFU were split into 2 groups—1 group received vehicle control treatment daily for 5 days, the other received 2 mg/kg Sar⁹, Met (O₂)¹¹-Substance P treatment intranasally commencing 1 day after infection and continuing thru Day 5. Similarly, animals infected with 10⁵ PFU were split into 2 groups—1 group received vehicle control treatment daily for 5 days, the other received 2 mg/kg Sar⁹, Met (O₂)¹¹-Substance P treatment intranasally commencing 1 day after infection and continuing thru Day 5.

As shown in the figures below animals challenged with the H5N1 used in this study were found to show robust temperature increases, weight loss, increased sickness scores (a measure of animal activity and responsiveness), and mortality. Animals infected and treated with 2 mg/kg Sar⁹, Met (O₂)¹¹-Substance P were observed to recover quicker from infection—sickness scores were reduced sooner, temperature increases (aka ‘fever’) normalized quicker, and more animals survived. Animals challenged with the lower dose of H5N1 (10³ PFU) were also found to have less weight loss when treated with 2 mg/kg Sar⁹, Met (O₂)¹¹-Substance P, however this effect was not observed in the animals challenged with the higher initial Influenza burden. It appears 2 mg/kg Sar⁹, Met (O₂)¹¹-Substance P reduced quantitative ‘illness’ approximately 2 days sooner than vehicle-treated controls. Furthermore, 2 mg/kg Sar⁹, Met (O₂)¹¹-Substance P increased survival of infected animals from 50% (n=8) to 80% (n=10)—an effect nearing statistical significance but limited by the sample size.

Animals treated with Substance P analogs such as Sar⁹, Met (O₂)¹¹-Substance P and the like are consistently observed to show less clinical symptomatology following infection with any one of multiple influenza strains. That is, the effects of Substance P analogs such as Sar⁹, Met (O₂)¹¹-Substance P are independent of the strain of influenza, which confirms the host-mediated mechanism of action of Substance P analogs such as Sar⁹, Met (O₂)¹¹-Substance P. In ‘seasonal’ influenza models, Substance P analogs such as Sar⁹, Met (O₂)¹¹-Substance P reduces viral titers of influenza found in both lungs and nares and reduces the clinical symptoms of the disease, such as pulmonary inflammation and weight loss. In a direct comparison with the “Gold Standard” treatment (Tamiflu®), Substance P analogs such as Sar⁹, Met (O₂)¹¹-Substance P is found to be more effective at reducing the symptoms of Influenza infection. In ‘highly-pathogenic’ or ‘avian’ influenza models, Substance P analogs such as Sar⁹, Met (O₂)¹¹-Substance P similarly reduces clinical symptoms of disease while greatly improving survival

While the invention has been described in connection with what is presently considered to be the most practical and preferred embodiment, it is to be understood that the invention is not to be limited to the disclosed embodiments but, on the contrary, is intended to cover various modifications and equivalent arrangements included within the spirit and scope of the appended claims, which scope is to be accorded the broadest interpretation so as to encompass all such modifications and equivalent structures as is permitted under the law.

1. A composition for preventing or ameliorating the onset of one or more symptoms of influenza, the composition comprising:
an effective amount of a substance P compound;
a carrier medium, wherein the carrier medium is one of an aqueous solution, a food grade organic liquid, an inert food grade solid and wherein the substance P compound is present in the carrier medium in an amount sufficient to provide a dose strength between 0.05 nanomolar and 10 micromolar, effective to trigger reduction of a symptom from a group including at least one of the following: neutrophil number, alveolar-capillary barrier membrane damage, pulmonary inflammation, weight loss, viral titers, mortality, fever, myalgia, wherein the substance P compound is a derivative of substance P selected from the group consisting of; Sar⁹, Met (O₂)¹¹-Substance P; [Met-OH¹¹]-substance P; [Met-OMe¹¹]-substance P; [Nle¹¹]-substance P; [Pro⁹]-substance P; [Sar⁹]-substance P; [Tyr⁸]-substance P; [p-Cl-Phe^{7,8}]-substance P.

2. (canceled)

3. (canceled)

4. The composition of claim 1 wherein the influenza is of H1N1, H3N2, H5N1 or H7N9 subtype.

5. The composition of claim 1 when in the composition is present as one of an oral, intravenous, intramuscular, intratracheal, sublingual or aerosolizable dose form.

6. The composition of claim 5 further comprising an effective amount of at least one antiviral agent selected from the group consisting of neuraminidase inhibitors, adamantanes and mixtures thereof.

7. The composition of claim 6 wherein the neuraminidase inhibitor is selected from the group consisting of oseltamivir, zanamivir and mixtures thereof and wherein the adamantine is selected from the group consisting of adamantadine, rimatadine and mixtures thereof.

8. (canceled)

9. The composition of claim 7 wherein the substance P compound is Sar⁹, Met (O₂)¹¹-Substance P.

10. (canceled)

11. A method of treating a patient exhibiting influenza symptoms, comprising:
administering an effective amount of a bioactive agent that includes one of the following: Substance P; Sar⁹, Met (O₂)¹¹-Substance P; [Met-OH¹¹]-substance P; [Met-OMe¹¹]-substance P; [Nle¹¹]-substance P; [Pro⁹]-substance P; [Sar⁹]-substance P; [Tyr⁸]-substance P; [p-Cl-Phe^{7,8}]-substance P; to an individual who has been exposed to influenza, whereby the individual exhibits reduction of a symptom from a group including at least one of the following: neutrophil number, alveolar-capillary barrier membrane damage, pulmonary inflammation, weight loss, viral titers, mortality, fever, myalgia.

12. The method of claim 11 wherein Sar⁹, Met (O₂)¹¹-Substance P is administered.

13. The method of claim 12 wherein the step of administering is performed by one of the following: inhalation of an aerosol, intramuscular delivery, sublingual delivery.

14. (canceled)

15. (canceled)

16. The method of claim 12 wherein the step of administering is performed by oral delivery of a dose form, wherein the dose form is one of a pill, tablet, powder or granulated material.

17. A method of protecting an individual prior to or after exposure to a patient with influenza from developing influenza, comprising the step of administering to the individual an effective amount of an agent selected from the group consisting of: Sar⁹, Met (O₂)¹¹-Substance P; [Met-OH¹¹]-substance P; [Met-OMe¹¹]-substance P; [Nle¹¹]-substance P; [Pro⁹]-substance P; [Sar⁹]-substance P; [Tyr⁸]-substance P; [p-Cl-Phe^{7,8}]-substance P.

18. The method of claim 17 wherein the agent is Sar⁹, Met (O₂)¹¹-Substance P.

19. The method of claim 18 wherein the step of administering is performed by one of the following: inhalation of an aerosol, intramuscular delivery, sublingual delivery.

20. (canceled)

21. (canceled)

22. (canceled)
